SEQUENCE LISTING (5 sequences)

SEQ ID NO: 1 - protein, 24 aa, Artificial Sequence
  CD8a transmembrane sequence
IYIWAPLAGTCGVLLLSLVITLYC

SEQ ID NO: 2 - protein, 112 aa, Artificial Sequence
  CD3z intracellular sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 42 aa, Artificial Sequence
  4-IBB (CD-137) intracellular sequence
KRGRKKLLYIFKQPFMRPVQTTQEEDGCSCRFPEEEEGGCEL

SEQ ID NO: 4 - protein, 19 aa, Artificial Sequence
  CD8a signal sequence
MAWVWTLLFLMAAAQSIQA

SEQ ID NO: 5 - protein, 45 aa, Artificial Sequence
  CD8a hinge region sequence
TSTPAPRPPTPAPTIASQPLSLRPEACRPAAGGAVHTRGLDFACD

(19) United States
(12) Patent Application Publication
(10) Pub. No.: US 2021/0355442 A1
(43) Pub. Date: Nov. 18, 2021

(54) GAMMA DELTA T CELLS AND USES THEREOF

(71) Applicant: Janssen Biotech, Inc., Horsham, PA (US)

(72) Inventors: Rajkumar GANESAN, Blue Bell, PA (US); Iqbal S. GREWAL, Newtown, PA (US); Sanjaya SINGH, Blue Bell, PA (US)

(21) Appl. No.: 17/190,566

(22) Filed: Mar. 3, 2021

Related U.S. Application Data
(60) Provisional application No. 62/984,445, filed on Mar. 3, 2020.

(51) Int. Cl. C12N 5/0783; A61K 35/17; C07K 14/705; C07K 14/725; C07K 16/30

(52) U.S. Cl. CPC C12N 5/0636; A61K 35/17; C07K 14/70517; C07K 14/7051; C07K 2319/02; C07K 16/30; C12N 2501/2302; C12N 2501/998; C07K 2319/03; C07K 14/70578

(57) ABSTRACT

Provided are methods of expanding and isolating γδ T cells from human peripheral blood mononuclear cells (PBMCs). Also provided are isolated γδ T cells, CAR-γδ T cells, and methods of using the same.

### CROSS-REFERENCE TO RELATED APPLICATIONS

This application claims priority to U.S. Provisional Application No. 62/984,445, filed Mar. 3, 2020, the disclosure of which is herein incorporated by reference in its entirety.

### FIELD OF THE INVENTION

This invention relates to the expansion and isolation of γδ T cells and the use of the γδ T cells to express a chimeric antigen receptor for adoptive T cell therapy.

### REFERENCE TO SEQUENCE LISTING SUBMITTED ELECTRONICALLY

This application contains a sequence listing, which is submitted electronically via EFS-Web as an ASCII formatted sequence listing with a file name “688097.898US Sequence Listing” and a creation date of Feb. 20, 2020 and having a size of 3 kb. The sequence listing submitted via EFS-Web is part of the specification and is herein incorporated by reference in its entirety.

### BACKGROUND OF THE INVENTION

The genetic engineering of T cells to specifically engage and kill tumor cells in a target-specific manner has resulted in the establishment of new therapeutic options for cancer patients, referred to as engineered T cell therapy. This targeting is typically brought about by genetically manipulating patient-derived T cells with a recombinant DNA molecule that encodes a chimeric antigen receptor (CAR). CARs are synthetic receptors comprising an extracellular targeting domain that is linked to a linker peptide, a transmembrane (TM) domain, and one or more intracellular signaling domains.

Traditionally, the extracellular domain consists of a single chain Fv fragment of an antibody (scFv) that is specific for a given tumor-associated antigen (TAA) or cell surface target. The extracellular scFv domain confers the tumor specificity of the CAR, while the signaling domains activate the T cell upon TAA/target engagement. These engineered T cells (CAR-T cells) are re-infused into cancer patients, where they specifically engage and kill cells expressing the TAA target of the CAR (Maus et al., Blood. 2014 Apr 24;123(17):2625-35; Curran and Brentjens, J Clin Oncol. 2015 May 20;33(15):1703-6).

Autologous, patient-specific CAR-T therapy has emerged as a powerful and potentially curative therapy for cancer, especially for CD19-positive hematological malignancies. However, the development of CAR-T technology and its wider application is partly limited due to number of key shortcomings including a) an inefficient anti-tumor response in solid tumors, b) limited penetration and susceptibility of adoptively transferred CAR T cells to an immunosuppressive tumor microenvironment (TME), c) poor persistence of CAR-T cells in vivo, d) serious adverse events in the patients including cytokine release syndrome (CRS) and graft-versus-host disease (GVHD) mediated by the CAR-T, and e) the time required for manufacturing.

To circumvent major issues with the current CAR-T approaches, an alternative CAR-T strategy comprising the generation of universal allogeneic CAR-T cells would be beneficial.

### BRIEF SUMMARY OF THE INVENTION

In one general aspect, provided is a method of expanding and isolating γδ T cells from human peripheral blood mononuclear cells (PBMCs). The methods comprise (a) obtaining human PBMCs; (b) culturing the human PBMCs in a culture media comprising interleukin-2 (IL-2) and an anti-TCR Vγ9 monoclonal antibody or antigen binding fragment thereof to expand the γδ T cells; and (c) isolating the γδ T cells.

In certain embodiments, the concentration of the IL-2 is about 50 IU/mL to about 5000 IU/mL. The concentration of IL-2 can, for example, be about 100 IU/mL to about 1000 IU/mL. In certain embodiments, the IL-2 is recombinant human IL-2 (rhIL-2). In certain embodiments, the γδ T cell is a Vγ9⁺ γγ T cell or a Vγ9⁻ γδ T cell. In certain embodiments, the γδ T cells are isolated by flow cytometry, magnetic separation, and negative selection.

Also provided are isolated γδ T cells produced by the methods of the invention. Also provided are methods of generating a chimeric antigen receptor (CAR)-γδ T cell. The methods comprise (a) obtaining an isolated γδ T cell of the invention; (b) contacting the γδ T cell with a nucleic acid encoding a chimeric antigen receptor (CAR), the CAR comprising (i) an extracellular domain; (ii) a transmembrane domain; and (iii) an intracellular signaling domain, wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

In certain embodiments, the CAR comprises (i) an extracellular domain comprising an antigen binding domain and/or an antigen binding fragment; (ii) a transmembrane domain comprising a CD8α transmembrane domain; (iii) an intracellular signaling domain comprising a CD3ζ or 4-1BB intracellular domain; (iv) a signal peptide comprising a CD8α signal peptide; and (v) a hinge region comprising a CD8α hinge region.

In certain embodiments, the CAR comprises (i) the transmembrane domain having an amino acid sequence at least 90% identical to SEQ ID NO:1; (ii) the intracellular domain having an amino acid sequence at least 90% identical to SEQ ID NO:2 or SEQ ID NO:3; (iii) the signal peptide having an amino acid sequence at least 90% identical to SEQ ID NO:4; and (iv) the hinge region having an amino acid sequence at least 90% identical to SEQ ID NO:5.

In certain embodiments, the extracellular domain comprises an antigen binding domain and/or an antigen binding fragment that specifically binds a tumor antigen.

Also provided are CAR-γδ T cells produced by the methods of the invention.

In certain embodiments, provided is a pharmaceutical composition comprising the CAR-γδ T cell of the invention and a pharmaceutically acceptable carrier.

Also provided are methods of treating or preventing a disease or condition in a subject in need thereof. The methods comprise administering a therapeutically effective amount of the pharmaceutical composition of the invention. In certain embodiments, the disease or condition is cancer. The cancer can, for example, be selected from a solid cancer or a liquid cancer. The cancer can be, but is not limited to, a cancer selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), a Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In certain embodiments, the disease or condition is an autoimmune disease. The autoimmune disease can be, but is not limited to, an autoimmune disease selected from the group consisting of alopecia, amyloidosis, ankylosing spondylitis, Castleman disease (CD), celiac disease, crohn's disease, endometriosis, fibromyalgia, glomerulonephritis, Graves' disease, Guillain-Barre syndrome, IgA nephropathy, lupus, lyme disease, Meniere;s disease, multiple sclerosis, narcolepsy, neutropenia, psoriasis, psoriatic arthritis, rheumatoid arthritis, sarcoidosis, scleroderma, type 1 diabetes, ulcerative colitis, and vitiligo.

Also provided are methods of producing a pharmaceutical composition comprising a CAR-γδ cell, wherein the methods comprise combining the CAR-γδ T cell of the invention with a pharmaceutically acceptable carrier to obtain the pharmaceutical composition.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1B show experimental and flow cytometry gating strategy for expanding and identifying Vγ9⁺ γδ T cells from whole PBMCs. FIG. 1A shows a brief layout of experiment design for anti-TCR Vγ9 mAb mediated expansion of γδ T-cells from whole PBMCs. FIG. 1B shows a schematic depiction of the flow cytometry gating strategy to identify the Vγ9⁺γδ T-cells among whole PBMCs or γδ T-cells.

FIGS. 2A-2B show anti-TCRVγ9 mAb mediated expansion of γδ T-cells from whole PBMCs. FIG. 2A shows flow cytometry plots for anti-TCRVγ9 mAb mediated expansion of γδ T cells from whole PBMCs. Briefly, a half million whole PBMCs were seeded into a well of 24-well plate pre-coated with anti-TCR Vγ9 mAb (clones B3, 7A5 and IMMU 360) and cultured in the presence 200 IU IL-2 and 2 μg/mL CD28 for 15 days. Numbers in quadrants of representative FACS plots show the frequency of αβ and γδ T-cells among total live PBMCs on day 0, 11 and 15 of the culture period. FIG. 2B shows a graph demonstrating the fold-expansion of TCR γδ T cells. Bars mirror the fold expansion (mean frequency±SD) of γδ T-cells among total live PBMCs, that were expanded either in the presence of plate bound anti-TCR Vγ9 mAb clones (B3, 7A5 and IMMU 360) or Zoledronic acid, on day 0, 11 and 15 of the culture period. n=2 donors from a single experiment.

FIGS. 3A-3B show anti-TCRVγ9 mAb mediated selective expansion of Vγ9⁺ cells among whole γδ T-cells. FIG. 3A shows flow cytometry plots for anti-TCRVγ9 mAb mediated selective expansion of Vγ9⁺ cells from whole γδ T-cells. Briefly, a half million whole PBMCs were seeded into a well of 24-well plate pre-coated with anti-TCR Vγ9 mAb (clones B3, 7A5 and IMMU 360) and cultured in the presence 200 IU IL-2 and 2 μg/mL CD28 for 15 days. Numbers above gates in representative FACS plots depict the frequency of Vγ9⁻ and Vγ9⁺ cells among total γδ T-cells on day 0, 11 and 15 of the culture period. FIG. 3B shows a graph demonstrating the percentage of Vγ9⁺ cells among γδ T-cells. Bars reflect the mean frequency (±SD) of Vγ9⁺ γδ T-cells among total γδ T-cells, that were expanded either in the presence of plate bound anti-TCR Vγ9 mAb clones (B3, 7A5 and IMMU 360) or Zoledronic acid, on day 0, 11 and 15 of the culture period. n=2 donors from a single experiment.

FIGS. 4A-4B show Vγ9⁺ γδ T-cells expanded either by anti-TCR Vγ9 mAbs or Zoledronic acid demonstrate similar activation and transduction abilities. FIG. 4A shows FACS plots at day 0 and day 15 for Vγ9⁺ γδ T-cells expanded either by anti-TCR Vγ9 mAbs or Zoledronic acid. Numbers above gates in representative FACS plots reflect the frequency of CD69⁺ cells among Vγ9⁺ γδ T-cells, from day 0 and day 15 of the culture, that were either expanded by anti-TCR Vγ9 mAb clones (B3, 7A5, and IMMU 360) or with Zoledronic acid. FIG. 4B shows a graph demonstrating percentage of CD69⁺ cells among Vγ9⁺ γδ T-cells. Bars depict the mean frequency (±SD) of CD69⁺ cells among Vγ9⁺ γδ T-cells that were expanded either by anti-TCR Vγ9 mAb clone (B3, 7A5, IMMU360) or Zoledronic acid on day 0, 11 and 15 of the culture. n=2 donors from a single experiment.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=-4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell (e.g., a γδ T cell), a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “immune cell” or “immune effector cell” refers to a cell that is involved in an immune response, e.g., in the promotion of an immune effector response. Examples of immune cells include T cells, B cells, natural killer (NK) cells, mast cells, and myeloid-derived phagocytes. According to particular embodiments, the engineered immune cells are T cells (e.g., γδ T cells), and are referred to as CAR-T cells (e.g., CAR γδ T cells) because they are engineered to express CARs of the invention.

As used herein, the term “engineered immune cell” refers to an immune cell, also referred to as an immune effector cell, that has been genetically modified by the addition of extra genetic material in the form of DNA or RNA to the total genetic material of the cell. According to embodiments herein, the engineered immune cells have been genetically modified to express a CAR construct according to the invention. Methods of expanding and purifying γδ T cells

Provided herein are γδ T cell-based allogenic off-the-shelf CAR-T cell products. Use of γδ T cells allows for the development of a high-quality product combining the inherent versatile nature of the γδ T cell with their highly potent cytolytic functions to reduce the risk of tumor escape. This approach can help to reduce the side effects mediated by CRS/GVHD and prevent long-term autoimmunity while providing excellent efficacy, particularly, in solid tumors. γδ T cells are abundant in the blood with good markers for sorting and can easily be activated and expanded in large numbers by well-defined ligands. Since γδ T cell recognition is independent of MHC, γδ T cells do not participate in GVHD, and there is no risk of allogeneic-recognition, γδ T cells can serve as an allogenic source of CAR-T for a broader population of patients. Off the shelf products can have several advantages including consistent quality, cells can be sorted for 100% transductions, no limitations on the dose size, reduced cost and significant time savings for the patients to initiate the treatment.

According to particular aspects, the invention provides methods of expanding and isolating γδ T cells from human peripheral blood mononuclear cells (PBMCs). In one general aspect, the methods comprise (a) obtaining human PBMCs; (b) culturing the human PBMCs in a culture media comprising interleukin-2 (IL-2) and an anti-TCR Vγ9 monoclonal antibody or antigen binding fragment thereof to expand the γδ T cells; and (c) isolating the γδ T cells. In certain embodiments, the γδ T cell is a Vγ9⁺ γδ T cell.

Also provided are isolated γδ T cells, including isolated Vγ9⁺ γδ T cells, produced by the methods of the invention.

In certain embodiments, the concentration of the IL-2 is about 50 IU/mL to about 5000 IU/mL. The concentration of IL-2 can, for example, be about 50 IU/mL to about 4000 IU/mL, about 50 IU/mL to about 3000 IU/mL, about 50 IU/mL to about 2000 IU/mL, about 50 IU/mL to about 1000 IU/mL, about 50 IU/mL to about 500 IU/mL, about 50 IU/mL to about 250 IU/mL, about 100 IU/mL to about 5000 IU/mL, about 100 IU/mL to about 4000 IU/mL, about 100 IU/mL to about 3000 IU/mL, about 100 IU/mL to about 2000 IU/mL, about 100 IU/mL to about 1000 IU/mL, about 100 IU/mL to about 500 IU/mL, about 250 IU/mL to about 5000 IU/mL, about 250 IU/mL to about 4000 IU/mL, about 250 IU/mL to about 3000 IU/mL, about 250 IU/mL to about 2000 IU/mL, about 250 IU/mL to about 1000 IU/mL, about 500 IU/mL to about 5000 IU/mL, about 500 IU/mL to about 4000 IU/mL, about 500 IU/mL to about 3000 IU/mL, about 500 IU/mL to about 2000 IU/mL, about 500 IU/mL to about 1000 IU/mL, about 1000 IU/mL to about 5000 IU/mL, about 1000 IU/mL to about 4000 IU/mL, about 1000 IU/mL to about 3000 IU/mL, about 1000 IU/mL to about 2000 IU/mL, about 2000 IU/mL to about 5000 IU/mL, about 2000 IU/mL to about 4000 IU/mL, about 2000 IU/mL to about 3000 IU/mL, or any concentration in between. In certain embodiments, the concentration of IL-2 is 50, 100, 250, 500, 1000, 1500, 2000, 2500, 3000, 3500, 4000, 4500, or 5000 IU/mL. In certain embodiments, the IL-2 is recombinant human IL-2 (rhIL-2).

Also provided are methods of generating a chimeric antigen receptor (CAR)-γδ T cell. The methods comprise (a) obtaining an isolated γδ T cell of the invention; (b) contacting the γδ T cell with a nucleic acid encoding a chimeric antigen receptor (CAR), the CAR comprising (i) an extracellular domain; (ii) a transmembrane domain; and (iii) an intracellular signaling domain, wherein the CAR optionally further comprises a signal peptide at the amino terminus and a hinge region connecting the extracellular domain and the transmembrane domain, and wherein contacting the γδ T cell with the nucleic acid encoding the CAR generates a CAR-γδ T cell.

Thus, in certain embodiments, the isolated γδ T cells can comprise an isolated polynucleotide encoding a CAR or a vector comprising the isolated polynucleotide encoding the CAR. The immune cells comprising the isolated polynucleotides and/or vectors can be referred to as “engineered immune cells.” Preferably, the engineered immune cells are derived from a human (are of human origin prior to being made recombinant). The engineered immune cells can, for example, be T cells, and in particular are γδ T cells isolated by the methods described herein. In certain embodiments, the γδ T cells are Vγ9⁺ γδ T cells isolated by the methods described herein.

γδ T cells, including Vγ9⁺ γδ T cells, can be expanded and isolated utilizing the methods disclosed herein. Immune cells can additionally be isolated by methods known in the art, including commercially available methods (see, e.g., Rowland Jones et al., Lymphocytes: A Practical Approach, Oxford University Press, NY (1999)). Sources for immune cells or precursors thereof include, but are not limited to, peripheral blood (e.g., peripheral blood mononuclear cells (PBMCs)), umbilical cord blood, bone marrow, or other sources of hematopoietic cells. Various techniques can be employed to separate the cells to isolated or enrich desired immune cells. For instance, negative selection methods can be used to remove cells that are not the desired immune cells. Additionally, positive selection methods can be used to isolated or enrich for the desired immune cells or precursors thereof, or a combination of positive and negative selection methods can be employed. If a particular type of cell is to be isolated, e.g., a particular T cell, various cell surface markers or combinations of markers (e.g., CD3, CD4, CD8, CD34) can be used to separate the cells. In certain embodiments, the γδ T cells are isolated by flow cytometry, magnetic separation, and negative selection.

The γδ T cells can be autologous or non-autologous to the subject to which they are administered in the methods of treatment of the invention. Autologous cells are isolated from the subject to which the engineered immune cells recombinantly expressing the CAR are to be administered. Alternatively, allogeneic cells from a non-autologous donor that is not the subject can be used. In the case of a non-autologous donor, the cells are typed and matched for human leukocyte antigen (HLA) to determine the appropriate level of compatibility. For both autologous and non-autologous cells, the cells can optionally be cryopreserved until ready for use.

According to particular embodiments, the method of making the engineered immune cells comprises transfecting or transducing immune effector cells isolated from an individual such that the immune effector cells express one or more CAR(s) according to embodiments of the invention. Methods of preparing immune cells for immunotherapy are described, e.g., in WO2014/130635, WO2013/176916 and WO2013/176915, which are incorporated herein by reference. Individual steps that can be used for preparing engineered immune cells are disclosed, e.g., in WP2014/039523, WO2014/184741, WO2014/191128, WO2014/184744 and WO2014/184143, which are incorporated herein by reference.

In a particular embodiment, the immune effector cells, such as γδ T cells, are genetically modified with CARs of the invention (e.g., transduced with a viral vector comprising a nucleic acid encoding a CAR) and then are activated and expanded in vitro.

In various embodiments, T cells can be activated and expanded before or after genetic modification to express a CAR, using methods as described, for example, in U.S. Pat. Nos. 6,352,694, 6,534,055, 6,905,680, 6,692,964, 5,858,358, 6,887,466, 6,905,681, 7,144,575, 7,067,318, 7,172,869, 7,232,566, 7,175,843, 5,883,223, 6,905,874, 6,797,514, 6,867,041, US2006/121005, which are incorporated herein by reference. T cells can be expanded in vitro or in vivo. Generally, the T cells of the invention can be expanded by contact with a surface having attached thereto an agent that stimulates a CD3/TCR complex-associated signal and a ligand that stimulates a co-stimulatory molecule on the surface of the T cells. As non-limiting examples, T cell populations can be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore, or by activation of the CAR itself. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include, e.g., an appropriate media (e.g., Minimal Essential Media or RPMI Media 1640 or, X-vivo 5 (Lonza)) that can contain factors necessary for proliferation and viability, including serum (e.g., fetal bovine or human serum), cytokines, such as IL-2, IL-7, IL-15, and/or IL-21, insulin, IFN-γ, GM-CSF, TGFβ and/or any other additives for the growth of cells known to the skilled artisan. In other embodiments, the T cells can be activated and stimulated to proliferate with feeder cells and appropriate antibodies and cytokines using methods such as those described in U.S. Pat. Nos. 6,040,177, 5,827,642, and WO2012129514, which are incorporated herein by reference.

Chimeric Antigen Receptors (CARs)

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane.

As used herein, the term “intracellular T cell receptor-activating signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by a T cell that provides the primary cytoplasmic signaling sequence(s) that regulate primary activation of the T cell receptor (TCR) complex in a stimulatory way for at least some aspect of the T cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain general aspects, provided herein are methods of generating a chimeric antigen receptor (CAR) γδ T cell. The methods can comprise (a) obtaining an isolated γδ T cell of the invention; (b) contacting the γδ T cell with a nucleic acid encoding a chimeric antigen receptor (CAR), the CAR comprising (i) an extracellular domain; (ii) a transmembrane domain; and (iii) an intracellular signaling domain, wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

In certain embodiments, the extracellular domain comprises an antigen binding domain and/or an antigen binding fragment. In certain embodiments, the antigen binding fragment is a Fab, a Fab′, a F(ab′)2, an Fv, a single-chain variable fragment (scFv), a minibody, a diabody, a single-domain antibody (sdAb), a light chain variable domain (VL), or a variable domain (VHH) of a camelid antibody. In certain embodiments, the antigen binding fragment is a single-chain variable fragment (scFv).

In certain embodiments, the antigen binding domain and/or antigen binding fragment can, for example, specifically bind a tumor antigen. Any suitable tumor antigen for binding by an antibody or antigen binding fragment can be chosen based on the type of tumor and/or cancer exhibited by the subject to be treated. Suitable antigens include, but are not limited to, mesothelin (MSLN), prostate specific membrane antigen (PSMA), prostate stem cell antigen (PCSA), B-cell maturation antigen (BCMA or BCM), G-protein coupled receptor family C group 5 member D (GPRCSD), Interleukin-1 receptor accessory protein (IL1RAP), delta-like 3 (DLL3), carbonic anhydrase IX (CAIX), carcinoembryonic antigen (CEA), CDS, CD7, CD10, CD19, CD20, CD22, CD30, CD33, CD34, CD38, CD41, CD44, CD49f, CD56, CD74, CD123, CD133, CD138, epithelial glycoprotein-2 (EGP 2), epithelial glycoprotein-40 (EGP-40), epithelial adhesion molecule (EpCAM), folate-binding protein (FBP), fetal acetylcholine receptor (AChR), folate receptor α and β (FRα and β), ganglioside G2 (GD2), ganglioside G3 (GD3), human epidermal growth factor receptor 2 (HER-2/ERB2), epidermal growth factor receptor (EGFR), epidermal growth factor receptor vIII (EGFRvIII), ERB3, ERB4, human telomerase reverse transcriptase (hTERT), interleukin-13 receptor subunit alpha-2 (IL-13Rα2), k-light chain, kinase insert domain receptor (KDR), Lewis A (CA19.9), Lewis Y (LeY), L1 cell adhesion molecule (LICAM), melanoma-associated antigen 1 (melanoma antigen family A1, MAGE-A1), Mucin-16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor receptor (VEGFR), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), chondroitin sulfate proteoglycan-4 (CSPG4), DNAX accessory molecule (DNAM-1), ephrin type A receptor 2 (EpHA2), fibroblast associated protein (FAP), Gp100/HLA-A2, glypican 3 (GPC3), HA-1H, HERK-V, IL-11Rα, latent membrane protein (LMP1), neural cell-adhesion molecule (N-CAM/CD56), and trail receptor (TRAIL R).

In certain embodiments, the extracellular domain of the CAR is preceded by a signal peptide at the amino-terminus. Any suitable signal peptide can be used in the invention. The signal peptide can, for example, be derived from a natural, synthetic, semi-synthetic, or recombinant source. According to one embodiment, the signal peptide is a human CD8a signal peptide, a human CD3δ signal peptide, a human CD3ζ signal peptide, a human GMCSFR signal peptide, a human 4-1BB signal peptide, or a derivative thereof. According to particular embodiments, the signal peptide is a human CD8a signal peptide. The human CD8α signal peptide comprises an amino acid sequence at least 90% identical to SEQ ID NO:4, preferably the amino acid sequence of SEQ ID NO:4. The signal peptide can be cleaved by a signal peptidase during or after completion of translocation of the CAR to generate a mature CAR free of the signal peptide.

In certain embodiments, the CAR can further comprise a hinge region connecting the extracellular domain and the transmembrane domain. The hinge region functions to move the extracellular domain away from the surface of the engineered immune cell to enable proper cell/cell contact, binding to the target or antigen and activation (Patel et al.,

Gene Therapy 6:412-9 (1999)). Any suitable hinge region can be used in a CAR of the invention. The hinge region can be derived from a natural, synthetic, semi-synthetic, or recombinant source. According to particular embodiments, the hinge region of the CAR is a hinge region from a CD8α peptide. In particular embodiments, the hinge region comprises an amino acid sequence at least 90% identical to SEQ ID NO:5, preferably the amino acid sequence of SEQ ID NO:5.

A CAR of the invention comprises a transmembrane domain. Any suitable transmembrane domain can be used in a CAR of the invention. The transmembrane domain can be derived from a natural, synthetic, semi-synthetic, or recombinant source. According to some embodiments, the transmembrane domain is a transmembrane domain from a peptide selected from the group consisting of a CD8α peptide, a CD28 peptide, a CD4 peptide, a CD3ζ peptide, a CD2 peptide, a 4-1BB peptide, an OX40 peptide, an ICOS peptide, a CTLA-4 peptide, a PD-1 peptide, a LAG-3 peptide, a 2B4 peptide, a BTLA peptide, a GMCSFR peptide, and the like. In particular embodiments, the transmembrane domain is a CD8α transmembrane domain. The CD8a transmembrane domain can comprise an amino acid sequence at least 90% identical to SEQ ID NO:1, preferably the amino acid sequence of SEQ ID NO:1.

A CAR of the invention comprises an intracellular signaling domain. Any suitable intracellular domain can be used in a CAR of the invention. In particular embodiments, the entire intracellular signaling domain is used. In other particular embodiments, a truncated portion of the signaling domain that transduces the effector or signal is used. According to embodiments of the invention, the intracellular signaling domain generates a signal that promotes an immune effector function of the CAR-containing cell, e.g., a CAR-T cell, including, but not limited to, proliferation, activation, and/or differentiation. In particular embodiments, the signal promotes, e.g., cytolytic activity, helper activity, and/or cytokine secretion of the CAR-T cell. According to some embodiments, the intracellular signaling domain of the CAR comprises a signaling domain of an Fcγ receptor (FcγR), an FCC receptor (FcϵR), an Fcα receptor (FcαR), neonatal Fc receptor (FcRn), CD3, CD3ζ, CD3γ, CD3δ, CD3ϵ, CD4, CDS, CD8, CD21, CD22, CD28, CD32, CD40L (CD154), CD45, CD66δ, CD79α, CD79β, CD80, CD86, CD278 (also known as ICOS), CD247ζ, CD247η, DAP10, DAP12, FYN, LAT, Lck, MAPK, MHC complex, NFAT, NF-κB, PLC-γ, iC3β, C3δγ, C3δ, and Zap70. According to some embodiments, the intracellular signaling domain is selected from the group consisting of a signaling domain of CD3ζ, FcRγ, FcRβ, CD3γ, CD3δ, CD3ϵ, CD5, CD22, CD79α, CD79β, and CD66δ. In particular embodiments, the intracellular domain is a CD3ζ or 4-1BB intracellular domain. The CD3ζ or 4-1BB intracellular domain can comprise an amino acid sequence at least 90% identical to SEQ ID NO:2 or 3, respectively, preferably the amino acid sequence of SEQ ID NO:2 or 3, respectively.

According to particular embodiments, the intracellular signaling domain further comprises one or more co-stimulatory signaling domains. The co-stimulatory domain can, for example, comprise a signaling domain of a peptide selected from: 2B4/CD244/SLAMF4, 4-1BB/TNFSF9/CD137, B7-1/CD80, B7-2/CD86, B7-H1/PD-L1, B7-H2, B7-H3, B7-H4, B7-H6, B7-H7, BAFF-R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD48/SLAMF2, CD49a, CD49D, CD49f, CD53, CD58/LFA-3, CD69, CD7, CD8α, CD8β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7Rα, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46,

NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R,

VLA1, and VLA-6. In certain embodiments, the costimulatory domain is selected from the group consisting of a costimulatory domain of one or more of CD28, 4-1BB (CD137), CD27, OX40, CD27, CD40, PD-1, ICOS, lymphocyte function-associated antigen-1 (LFA-1), CD2, CD7, LIGHT, NKG2C, B7-H3, TNFRSF9, TNFRSF4, TNFRSF8, CD40LG, ITGB2, KLRC2, TNFRSF18, TNFRSF14, HAVCR1, LGALS9, CD83, and a ligand that specifically binds with CD83.

### Antigen-Binding Fragments

### Antibodies

The invention generally relates to CAR constructs comprising an antigen binding fragment. The antigen binding fragment can, for example, be an antibody or antigen binding fragment thereof that specifically binds a tumor antigen. The antigen binding fragments of the invention possess one or more desirable functional properties, including but not limited to high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

The antigen binding fragment can, for example, be an antibody or antigen binding fragment thereof that specifically binds a tumor antigen. Any suitable tumor antigen for binding by an antibody or antigen binding fragment can be chosen based on the type of tumor and/or cancer exhibited by the subject to be treated. Suitable antigens include, but are not limited to, mesothelin (MSLN), prostate specific membrane antigen (PSMA), prostate stem cell antigen (PCSA), carbonic anhydrase IX (CAIX), B-cell maturation antigen (BCMA or BCM), G-protein coupled receptor family C group 5 member D (GPRCSD), Interleukin-1 receptor accessory protein (IL1RAP), delta-like 3 (DLL3), carcinoembryonic antigen (CEA), CDS, CD7, CD10, CD19, CD20, CD22, CD30, CD33, CD34, CD38, CD41, CD44, CD49f, CD56, CD74, CD123, CD133, CD138, epithelial glycoprotein-2 (EGP 2), epithelial glycoprotein-40 (EGP-40), epithelial adhesion molecule (EpCAM), folate-binding protein (FBP), fetal acetylcholine receptor (AChR), folate receptor α and β (FRα and β), ganglioside G2 (GD2), ganglioside G3 (GD3), human epidermal growth factor receptor 2 (HER-2/ERB2), epidermal growth factor receptor (EGFR), epidermal growth factor receptor vIII (EGFRvIII), ERB3, ERB4, human telomerase reverse transcriptase (hTERT), interleukin-13 receptor subunit alpha-2 (IL-13Rα2), k-light chain, kinase insert domain receptor (KDR), Lewis A (CA19.9), Lewis Y (LeY), L1 cell adhesion molecule (LICAM), melanoma-associated antigen 1 (melanoma antigen family Al, MAGE-A1), Mucin-16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor receptor (VEGFR), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), chondroitin sulfate proteoglycan-4 (CSPG4), DNAX accessory molecule (DNAM-1), ephrin type A receptor 2 (EpHA2), fibroblast associated protein (FAP), Gp100/HLA-A2, glypican 3 (GPC3), HA-1H, HERK-V, IL-11Rα, latent membrane protein (LMP1), neural cell-adhesion molecule (N-CAM/CD56), and trail receptor (TRAIL R). The methods of expanding and isolating γδ T cells from the human PBMCs described herein comprise a step of culturing the human PBMCs in a culture media comprising interleukin-2 (IL-2) and an anti-TCR Vγ9 monoclonal antibody or antigen binding fragment thereof.

In certain embodiments, the anti-TCR Vγ9 monoclonal antibody or antigen binding fragment thereof is chimeric. In certain embodiments, the anti-TCR Vγ9 monoclonal antibody or antigen binding fragment thereof is human or humanized.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on the tumor antigen and the second epitope is located on PD-1, PD-L1, CTLA-4, EGFR, HER-2, CD19, CD20, CD33, CD3, and/or other tumor associated immune suppressors or surface antigens.

As used herein, an antigen binding domain or antigen binding fragment that “specifically binds to a tumor antigen” refers to an antigen binding domain or antigen binding fragment that binds a tumor antigen, with a KD of 1×10⁻⁷M or less, preferably 1×10⁻⁸M or less, more preferably 5×10⁻⁹ M or less, 1×10⁻⁹M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen binding domain or antigen binding fragment can be determined by using surface plasmon resonance, such as by using a biosensor system, e.g., a Biacore® system, or by using bio-layer interferometry technology, such as an Octet RED96 system.

The smaller the value of the KD of an antigen binding domain or antigen binding fragment, the higher affinity that the antigen binding domain or antigen binding fragment binds to a target antigen.

According to particular aspects, the invention relates to a CAR construct comprising an antigen binding fragment, wherein the antigen binding fragment is an antibody or antigen binding fragment that specifically binds a tumor antigen. The antibody or antigen binding fragment can, for example, be a Fab, a Fab′, a F(ab′)2, an Fv, a single-chain variable fragment (scFv), a minibody, a diabody, a single-domain antibody (sdAb), a light chain variable domain (VL), or a variable domain (V_{H}H) of a camelid antibody.

### Polynucleotides, Vectors, and Host Cells

In another general aspect, the invention relates to an isolated nucleic acid encoding a chimeric antigen receptor (CAR) of the invention. It will be appreciated by those skilled in the art that the coding sequence of a CAR can be changed (e.g., replaced, deleted, inserted, etc.) without changing the amino acid sequence of the protein. Accordingly, it will be understood by those skilled in the art that nucleic acid sequences encoding CARS of the invention can be altered without changing the amino acid sequences of the proteins.

In another general aspect, the invention relates to a vector comprising a CAR of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a CAR in the cell. Conventional cloning techniques or artificial gene synthesis can be used to generate a recombinant expression vector according to embodiments of the invention.

In another general aspect, the invention relates to a host cell comprising a vector of the invention and/or an isolated nucleic acid encoding a CAR of the invention. Any host cell known to those skilled in the art in view of the present disclosure can be used for recombinant expression of CARs of the invention. Suitable host cells include prokaryotes, yeast, mammalian cells, or bacterial cells. In some embodiments, the host cells are E. coli TG1 or BL21 cells (for expression of, e.g., a CAR, a scFv, or sdAb), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

### Pharmaceutical Compositions

In another general aspect, the invention relates to a pharmaceutical composition comprising an isolated polynucleotide of the invention, an isolated polypeptide of the invention, a host cell of the invention, and/or an engineered immune cell of the invention and a pharmaceutically acceptable carrier. The term “pharmaceutical composition” as used herein means a product comprising an isolated polynucleotide of the invention, an isolated polypeptide of the invention, a host cell of the invention, and/or an engineered immune cell of the invention together with a pharmaceutically acceptable carrier. Polynucleotides, polypeptides, host cells, and/or engineered immune cells of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

### Methods of Use

In another general aspect, the invention relates to a method of treating a disease or a condition in a subject in need thereof. The methods comprise administering to the subject in need thereof a therapeutically effective amount of an engineered immune cell and/or a pharmaceutical composition of the invention. In certain embodiments, the disease or condition is cancer. The cancer can, for example, be a solid or a liquid cancer. The cancer, can, for example, be selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

According to embodiments of the invention, the pharmaceutical composition comprises a therapeutically effective amount of an isolated polynucleotide, an isolated polypeptide, a host cell, and/or an engineered immune cell. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to an isolated polynucleotide, an isolated polypeptide, a host cell, an engineered immune cell, and/or a pharmaceutical composition of the invention a therapeutically effective amount means an amount of the isolated polynucleotide, the isolated polypeptide, the host cell, the engineered immune cell, and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the invention and/or the pharmaceutical compositions of the invention can be administered in any convenient manner known to those skilled in the art. For example, the cells of the invention can be administered to the subject by aerosol inhalation, injection, ingestion, transfusion, implantation, and/or transplantation. The compositions comprising the cells of the invention can be administered transarterially, subcutaneously, intradermaly, intratumorally, intranodally, intramedullary, intramuscularly, inrapleurally, by intravenous (i.v.) injection, or intraperitoneally. In certain embodiments, the cells of the invention can be administered with or without lymphodepletion of the subject.

The pharmaceutical compositions comprising cells of the invention expressing CARs of the invention can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the invention.

The cells of the invention and/or the pharmaceutical compositions of the invention can be administered in any physiologically acceptable vehicle. A cell population comprising cells of the invention can comprise a purified population of cells. Those skilled in the art can readily determine the cells in a cell population using various well known methods. The ranges in purity in cell populations comprising genetically modified cells of the invention can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the invention are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the invention are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

### EMBODIMENTS

This invention provides the following non-limiting embodiments.

Embodiment 1 is a method of expanding and isolating γδ T cells from human peripheral blood mononuclear cells (PBMCs), the method comprising:
a. obtaining human PBMCs;
b. culturing the human PBMCs in a culture media comprising interleukin-2 (IL-2) and an anti-TCR Vγ9 monoclonal antibody or antigen binding fragment thereof to expand the γδ T cells; and
c. isolating the γδ T cells.

Embodiment 2 is the method of embodiment 1, wherein the concentration of the IL-2 is about 50 IU/mL to about 5000 IU/mL.

Embodiment 3 is the method of embodiment 2, wherein the concentration of IL-2 is about 100 IU/mL to about 1000 IU/mL.

Embodiment 4 is the method of any one of embodiments 1-3, wherein the IL-2 is recombinant human IL-2 (rhIL-2).

Embodiment 5 is the method of any one of embodiments 1-4, wherein the anti-TCR Vγ9 monoclonal antibody or antigen binding fragment thereof is chimeric.

Embodiment 6 is the method of any one of embodiments 1-4, wherein the anti-TCR Vγ9 monoclonal antibody or antigen binding fragment thereof is human or humanized.

Embodiment 7 is the method of any one of embodiments 1-6, wherein the γδ T cell is a Vγ9⁺ γδ T cell or a Vγ9⁻ γδ T cell.

Embodiment 8 is the method of any one of embodiments 1-7, wherein the γδ T cells are isolated by flow cytometry, magnetic separation, and negative selection.

Embodiment 9 is an isolated γδ T cell produced by the method of embodiment 8.

Embodiment 10 is a method of generating a chimeric antigen receptor (CAR)-γδ T cell, the method comprising:
a. obtaining an isolated γδ T cell of embodiment 9;
b. contacting the γδ T cell with a nucleic acid encoding a chimeric antigen receptor (CAR), the CAR comprising:
   i. an extracellular domain;
   ii. a transmembrane domain; and
   iii. an intracellular signaling domain,
      wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

Embodiment 11 is the method of embodiment 10, wherein the CAR comprises:
i. an extracellular domain comprising an antigen binding domain and/or an antigen binding fragment;
ii. a transmembrane domain comprising a CD8a transmembrane domain;
iii. an intracellular signaling domain comprising a CD3ζ or 4-1BB intracellular domain;
iv. a signal peptide comprising a CD8a signal peptide; and
v. a hinge region comprising a CD8a hinge region.

Embodiment 12 is the method of embodiment 11, wherein the CAR comprises:
i. the transmembrane domain having an amino acid sequence at least 90% identical to SEQ ID NO:1;
ii. the intracellular domain having an amino acid sequence at least 90% identical to SEQ ID NO:2 or SEQ ID NO:3;
iii. the signal peptide having an amino acid sequence at least 90% identical to SEQ ID NO:4; and
iv. the hinge region having an amino acid sequence at least 90% identical to SEQ ID NO:5.

Embodiment 13 is the method of embodiment 11 or 12, wherein the extracellular domain comprises an antigen binding domain and/or an antigen binding fragment that specifically binds a tumor antigen.

Embodiment 14 is a CAR-γδ T cell produced by the method of any one of embodiments 10-13.

Embodiment 15 is a pharmaceutical composition comprising the CAR-γδ T cell of embodiment 14 and a pharmaceutically acceptable carrier.

Embodiment 16 is a method of treating or preventing a disease or condition in a subject in need thereof, the method comprising administering a therapeutically effective amount of the pharmaceutical composition of embodiment 15.

Embodiment 17 is the method of embodiment 16, wherein the disease or condition is cancer.

Embodiment 18 is the method of embodiment 17, wherein the cancer is selected from a solid cancer or a liquid cancer.

Embodiment 19 is the method of embodiment 18, wherein the cancer is selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), a Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 20 is the method of embodiment 16, wherein the disease is an autoimmune disease.

Embodiment 21 is the method of embodiment 20, wherein the autoimmune disease is selected from the group consisting of alopecia, amyloidosis, ankylosing spondylitis, Castleman disease (CD), celiac disease, crohn's disease, endometriosis, fibromyalgia, glomerulonephritis, Graves' disease, Guillain-Barre syndrome, IgA nephropathy, lupus, lyme disease, Meniere's disease, multiple sclerosis, narcolepsy, neutropenia, psoriasis, psoriatic arthritis, rheumatoid arthritis, sarcoidosis, scleroderma, type 1 diabetes, ulcerative colitis, and vitiligo.

Embodiment 22 is a method of producing a pharmaceutical composition comprising a CAR-γδ T cell, wherein the methods comprises combining the CAR-γδ T cell of embodiment 14 with a pharmaceutically acceptable carrier to obtain the pharmaceutical composition.

### EXAMPLES

Materials and Methods

Abbreviations

TABLE 1
Abbreviations

| Abbreviations | Definitions |
|---|---|
| mL | Microliter |
| mM | Micromolar |
| FMO | Fluorescence minus one |
| FACS | Fluorescence-activated cell sorting |
| FBS | Fetal bovine serum |
| PBMCs | Peripheral blood mononuclear cells |
| PBS | Phosphate buffered saline |
| RPMI | Roswell Park Memorial Institute Medium |
| SL. No | Serial number |
| mL | Millliliter |
| Min | Minute |
| RPM | Revolutions per minute |
| ° C. | Degree Celsius |
| IU | International Units |
| Zol | Zoledronic acid monohydrate |
| Complete RPMI medium | RPMI + 10% FBS + 1X Pen/Strep |
| Conc. | Concentration |
| CAR | Chimeric Antigen Receptor |
| LV | Lenti-virus |
| mAb | Monoclonal antibody |
| MOI | Multiplicity of Infection |
| IL-2 | Interleukin-2 |

### Methods

Isolation of PBMCs from Whole Blood

Heparinized blood was diluted by adding equal volumes of room temperature equilibrated Plain RPMI (RPMI+1% Pen Strep) medium. 30 ml of diluted blood was carefully layered onto 15 mL of LYMPHOPREP™ in a 50 mL falcon tube using a Steripette. Care was taken to prevent gradient disturbance while layering the blood onto LYMPHOPREP™. The 50 mL falcon tube was centrifuged 450×g at room temperature for 30 minutes. After centrifugation, the falcon tube was carefully removed from centrifuge, and the upper plasma layer was discarded without disturbing the Plasma-Buffy coat-LYMPHOPREP™ interface. The buffy coat from the Plasma-Buffy coat-LYMPHOPREP™ interface was transferred into a new 50 mL falcon tube containing 30 mL of room temperature equilibrated complete RPMI medium (RPMI+10% FBS +1% Pen/Strep) without disturbing the erythrocyte/granulocyte pellet. The 50 mL falcon tube was centrifuged at 350×g for 10 minutes at room temperature, and the resultant supernatant was discarded. Red blood cell (RBC) lysis was performed by resuspending the cell pellet in 1 mL of ACK lysis solution at room temperature for 5-8 min. After the incubation period, RBC lysis was stopped by adding 20 mL of complete RPMI medium. The falcon tube was centrifuged at 350×g for 10 min at room temperature, and the cell pellet was washed once with 35 mL of 1×PBS. The falcon tube was centrifuged at 350×g for 10 min at room temperature, and the cell pellet was resuspended in complete RPMI medium. The resuspended cells were counted on a hemocytometer. The PBMCs were either directly used for downstream applications or frozen down in medium (10% DMSO+90% FBS) at a cell density of 25×10⁶/mL.

Zoledronic Acid-Mediated Selective Expansion of Vγ9⁺ γδ T-Cells from Whole PBMCs

On day 0, PBMCs that were isolated, as described above, from the whole blood sample by density gradient centrifugation were counted, and the cell density was adjusted to 1.0×10⁶ cells/mL in complete RPMI medium (RPMI+10% FBS+1×Pen/Strep). Alternatively, PBMCs were obtained by quickly thawing a vial of frozen PBMCs and added to 49 mL of warm complete RPMI (RPMI+10% FBS+1×Pen/Strep) medium in a 50 mL falcon tube to dilute the freezing medium. The PBMCS were centrifuged at 1500 rpm for 5 min, and the cells were washed once by re-suspending them in 35 mL of complete RPMI medium (RPMI+10% FBS+1×Pen/Strep). The cell pellet was re-suspended in complete RPMI medium, and the cells were counted. Meanwhile, γδ T cell culture medium was prepared by supplementing complete RPMI medium with recombinant human IL-2 (rhIL-2) to a final conc of 200 IU/mL and Zoledronic acid to a final conc of 5 μM. The cell density was adjusted to 1×10⁶ cells/mL with the prepared γδ T cell culture medium. 0.5×10⁶ PBMCs were seeded in 0.5 mL of γδ T cell culture medium in a 24-well plate. On day 2, the 24-well plate was topped with 0.5 mL of fresh γδ T cell culture medium supplemented with rhIL-2 to a concentration of 400 IU/mL (the final concentration of rhIL-2 was 200 IU/mL). On day 5, the cells were centrifuged at 1500 rpm for 5 min. The PBMCs were cultured for a total of 15 days by constantly replenishing the medium with fresh complete RPMI medium containing 200 IU IL-2 on day 4, 6, 8, 11 and 14 of the culture period. Depending on the confluency, cells were initially shifted to a 6-well plate on day 6 and to a T-25 flask on day 11 of the culture. On day 11 and 15 of the culture period, cells were profiled with anti-TCR γδ, TCR αβ, and TCRVγ9 mAbs to enumerate the frequency of TCR γδ, TCR αβ, and TCR Vγ9⁺ γδ T-cells among whole PBMCs.

Anti-TCR Vγ9 mAb-Mediated Selective Expansion of Vγ9⁺ γδ T-Cells from Whole PBMCs

On day 0, a well of a 24-well plate was coated with purified anti-TCR Vγ9 mAb (Clones B3 (BD Biosciences; Franklin Lakes, N.J.), 7A5 (Abcam; Cambridge, United Kingdom), and IMMU360 (Beckman Coulter; Brea, Calif.) at a conc of lug/ml) in 0.5 mL PBS for overnight incubation at 4° C. On day 1, PBS was aspirated from the well without touching the bottom of the well. Half a million whole PBMCs, that were isolated as described above, were added to the well in 0.5 mL complete RPMI medium containing 200 IU IL-2 and CD28 (2 μg/mL). On day 2, the 24-well plate was topped with 0.5mL of fresh γδ T-cell culture medium supplemented with rhIL-2 to a concentration of 400 IU/mL (the final concentration of rhIL-2 was 200 IU/mL). PBMCs were cultured for a total of 15 days by constantly replenishing the medium with fresh complete RPMI medium containing 200 IU IL-2 on day 4, 6, 8, 11 and 14 of the culture period. Depending on the confluency, cells were initially shifted to a 6-well plate on day 6 and to a T-25 flask on day 11 of the culture. On day 11 and 15 of the culture period, cells were profiled with anti-TCR γδ, TCR αβ, and TCRVγ9 mAbs to enumerate the frequency of TCR γδ, TCRαβ, and TCR Vγ9⁺ γδ T-cells among whole PBMCs.

### Phenotypic Profiling of γδ T-Cells

### Surface Phenotypic Profiling

Cultured PBMCs were harvested, washed once with FACS buffer (1×PBS+2% FBS) and cells were counted using a hemocytometer. One hundred thousand PBMCs were centrifuged at 1500 rpm for 5 min in a 96-well V-bottom plate, and the supernatant was discarded. The cell pellet was re-suspended in 100 μL of 1×PBS containing 0.1 of Live/Dead fixable violet dead cell stain and 0.5 μL of human Trustain Fc block. The cells were incubated in the dark at 4° C. for 30 min. After incubation in the dark, the cells were centrifuged at 1500 rpm for 5 min, and the cell pellet was washed once with 200 μL of FACS buffer (PBS+2% FBS). After incubation, the cells were centrifuged at 1500 rpm for 5 min. At the end of incubation period, 100 μL of FACS buffer (PBS+2% FBS) was added, and the cells were centrifuged at 1500 rpm for 5 min. The cells were washed once with 200 μL of FACS buffer (1×PBS+2% FBS), and the cells were surface stained by incubating in 100 μL of FACS buffer (1×PBS+2% FBS) containing antibodies specific for TCR γδ, TCR Vγ9, and TCR αβ at 4° C. for 30 min. After the incubation period, 100 μL of FACS buffer (PBS+2% FBS) was added, and the cells were centrifuged at 1500 rpm for 5 min. The cells were washed twice with 200 μL of FACS buffer and re-suspended in 100 μL of FACS buffer. Cells were acquired on flow cytometer (Novocyte).

### Lenti Viral Transduction

PBMCs (0.5×10⁶ cells), which were cultured either on plate bound anti-TCR Vγ9 mAbs or with Zol for 15 days, were centrifuged at 1250 rpm for 5 min at RT. The cell pellet was washed with 1mL of sterile PBS and was centrifuged at 1250 rpm for 5 min. The cell pellet was resuspended in 500 μL of complete RPMI medium+100 μL of MAX Enhancer and 2 μL of TRANSDUX MAX™. 5 μL of 1M HEPES buffer (final conc 10 μM) was added to the resuspended cells. The PBMCs, HEPES buffer and TRANSDUX MAX™ mixture were combined and transferred to a well of a 24 well plate. Virus (Anti-PSMA CAR lentivirus or empty pLLV lentivirus particles) was added to the mixture at an MOI of 10. The cells were spinoculated by spinning them at 32° C. at 1350×g for 1.5 hours. After spinning the cells, 1mL of complete fresh RPMI medium was added, and then the contents were transferred to a sterile Eppendorf tube. The cells were centrifuged at 1250 rpm for 5 min, the supernatant was removed, and the cells were resuspended in 0.5 mL of fresh complete RPMI medium containing 50 IU IL-2. The cells were plated in a 24 well plate and incubated for 96 hours at 37° C. in a 5% CO₂ incubator. After 72 hours, 0.5 mL of fresh complete RPMI medium containing 50 IU IL-2 was added to the wells. After 96 hours of lenti viral transduction, the cells were harvested from the well, washed once, counted, and subjected to surface staining with antibodies against TCR γδ, TCR αβ, TCRVγ9 γδ and Rabbit anti camelid VHH antibody.

### Acquisition and Analysis

For surface staining, stained cells were acquired on a Novocyte Flow Cytometer with NovoExpress software. Data was transferred via general folder to a desktop with Flow Jo (version 10.3). GraphPad Prism (Version 7.02 or 8.0) was used to plot mean frequency±SD values.

### Results

### Example 1

### Selective Expansion of Vγ9⁺ γδ T-Cells by Anti-TCR Vγ9 Monoclonal Antibodies

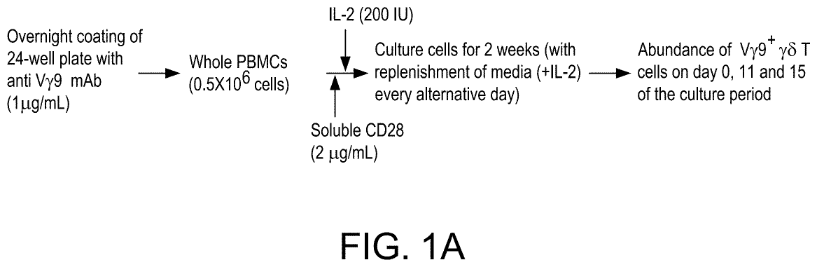

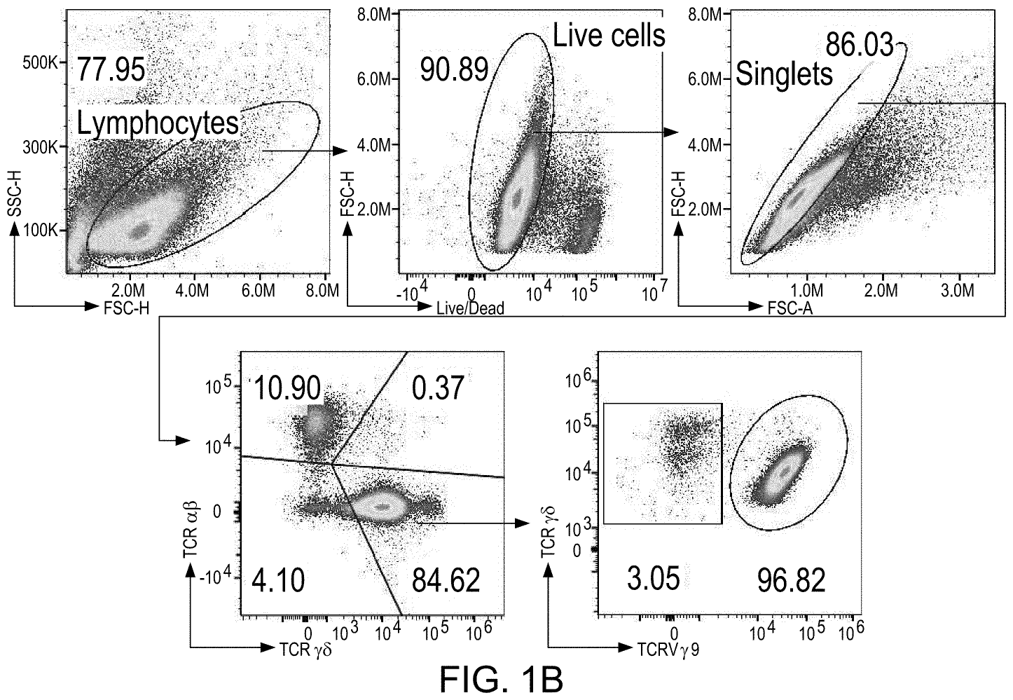

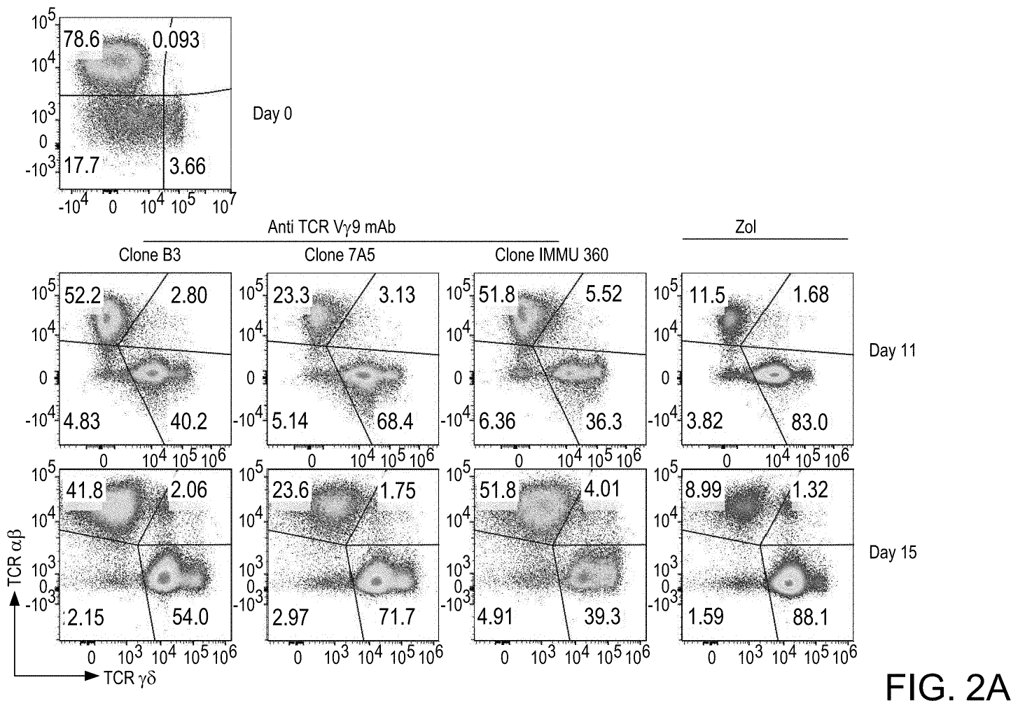

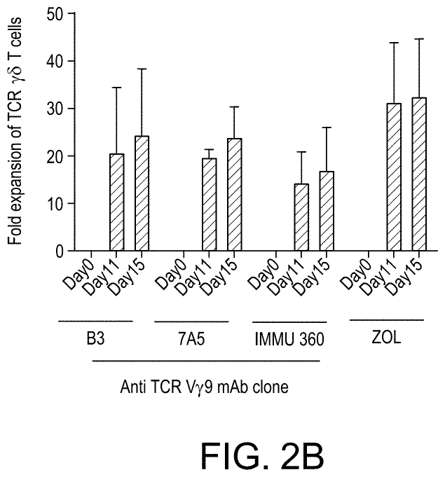

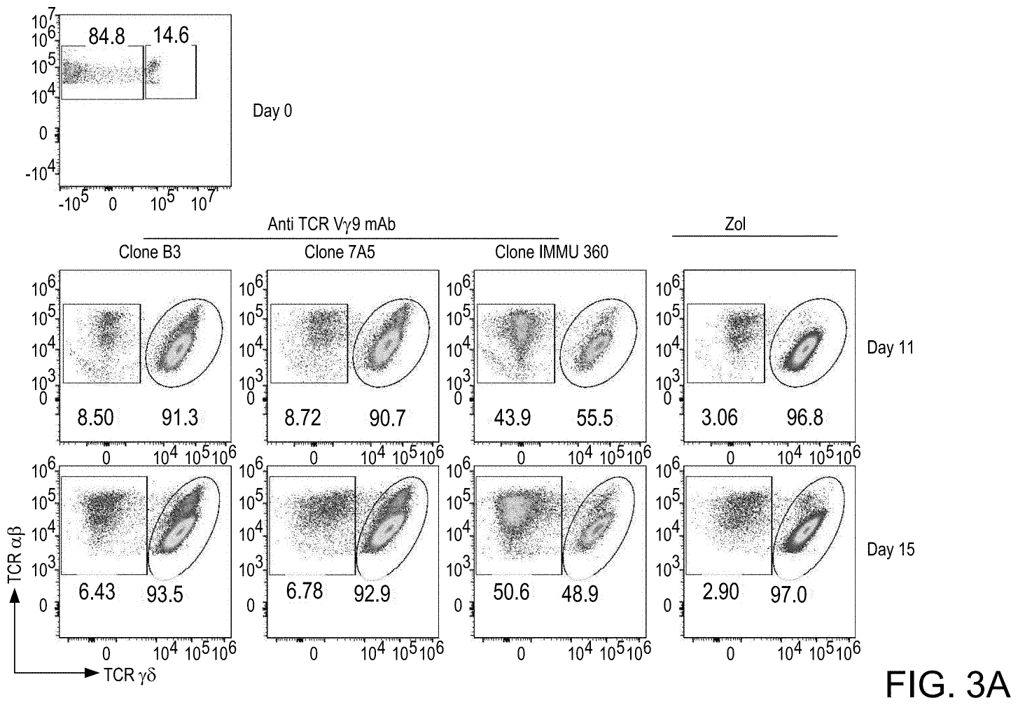

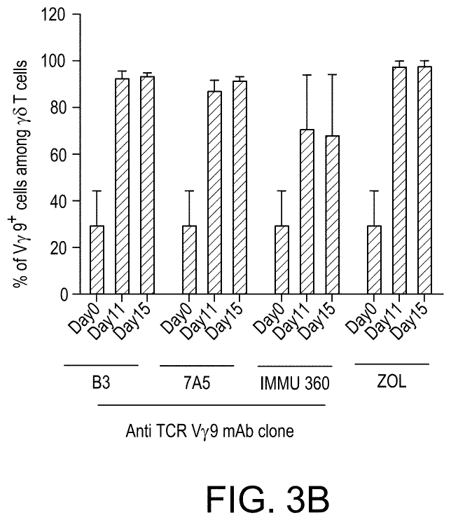

To selectively expand TCRVγ9⁺ γδ T-cells from whole PBMCs, half a million whole PBMCs were seeded into a well of a 24-well plate pre-coated with anti-TCR Vγ9 mAbs (clone B3, 7A5 and IMMU360) (FIG. 1A). In parallel, the same number of whole PBMCs were cultured in the presence of Zoledronic acid and IL-2 to selectively expand TCR Vγ9⁺ γδ T-cells from whole PBMCs, as described in U.S. application Ser. No. 16/691,812, filed on November 22, 2019. Frequency of γδ and αβ T-cells were enumerated on day 0, 11 and 15 of the culture period. To enumerate the frequency of γδ T-cells, initially, total live PBMCs were gated, excluding doublets, and the live cells were plotted against the TCR γδ and TCR αβ gates (FIG. 1B). To enumerate the frequency of TCRVγ9⁺ and TCRVγ9⁻ cells among total γδ T-cells, live γδ T-cells were plotted against TCR Vγ9. TCRVγ9⁺ TCRγδ⁺ double positive cells were considered as TCRVγ9⁺ γδ T-cells, and TCRγδ single positive cells were considered as TCRVγ9⁻ γδ T-cells (FIG. 1B). Compared to the frequency of TCRγδ T-cells among whole PBMCs on day 0 of the culture, a substantial expansion of Vγ9⁺ γδ T-cells was observed on day 11 and 15 of the culture period (FIG. 2A) from the wells coated with anti-TCR Vγ9 mAb clones B3, 7A5, and IMMU 360. On day 15 of the culture, fold (mean frequency±SD) expansion of TCR γδ T-cells mediated by anti-TCR Vγ9 mAb clones B3, 7A5, and IMMU 360 were 24.5, 23.9 and 17.0-fold, respectively, compared to 32.5-fold expansion mediated by Zol (FIG. 2B). Interestingly, anti-TCR Vγ9 mAb clones B3 and 7A5 selectively expanded Vγ9⁺, but not Vγ9⁻ γδ T-cells among whole γδ T-cells (FIGS. 3A and 3B). In contrast, clone IMMU 360 mediated either low expansion of TCR Vγ9⁺ γδ T-cells or favored expansion of Vγ9⁻ γδ T-cells (FIGS. 3A and 3B). Of note, frequency of TCRVγ9⁺ cells that makeup the entire γδ T cell compartment on day 15 of the culture with anti TCRVγ9 mAb clones B3, 7A5, and IMMU 360 were 94%, 92%, and 68% respectively, compared to 98% from Zol based expansion.

### Example 2

### Vγ9+ γδ T-Cells Expanded Either by Anti-TCR Vγ9 mAb or Zol Exhibit Similar Activation and Transduction Abilities

To understand whether TCR Vγ9⁺ γδ T-cells that were expanded either by TCR Vγ9 mAbs (clone B3, 7A5, and IMMU 360) or by Zol exhibit similar activation profiles, the activation status of the cells was probed by measuring the surface expression of CD69 on day 0, 11 and 15 of the culture.

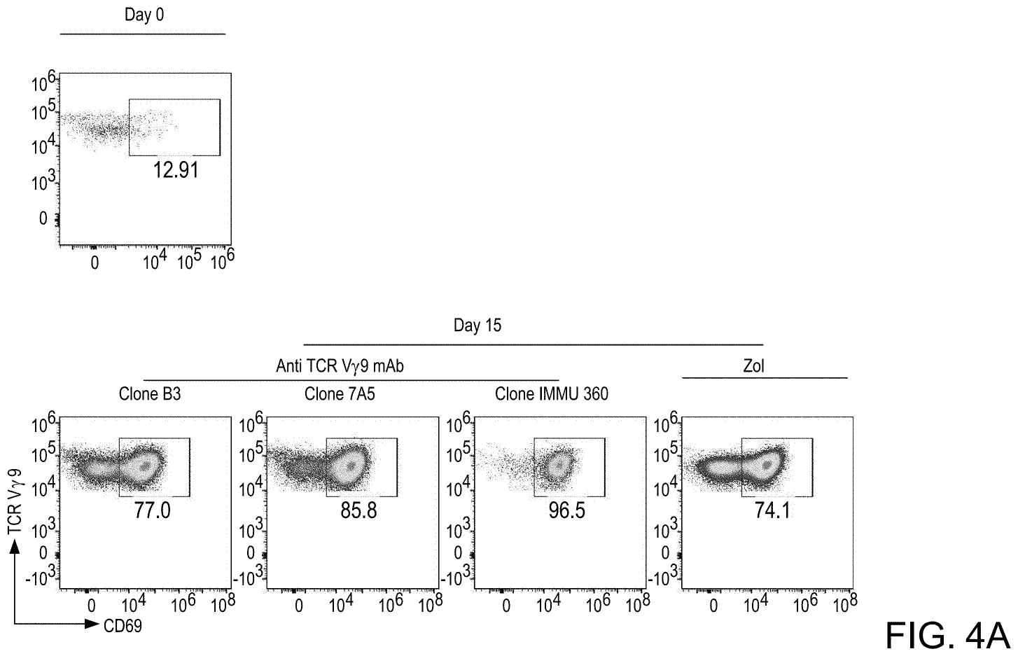

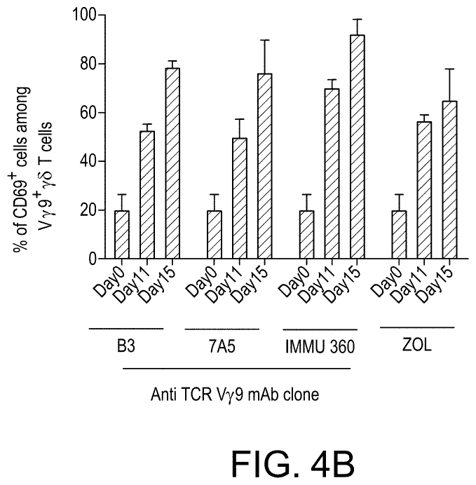

Interestingly, Vγ9⁺ γδ T-cells coming from both expansion conditions, anti-TCRVγ9 mAb-mediated and Zol-mediated, did not show any difference in their surface expression of CD69, a classical marker for lymphocyte activation and tissue retention (FIGS. 4A and 4B).

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method of expanding and isolating γδ T cells from human peripheral blood mononuclear cells (PBMCs), the method comprising:
a. obtaining human PBMCs;
b. culturing the human PBMCs in a culture media comprising interleukin-2 (IL-2) and an anti-TCR Vγ9 monoclonal antibody or antigen binding fragment thereof to expand the γδ T cells; and
c. isolating the γδ T cells.

2. The method of claim 1, wherein the concentration of the IL-2 is about 50 IU/mL to about 5000 IU/mL.

3. The method of claim 2, wherein the concentration of IL-2 is about 100 IU/mL to about 1000 IU/mL.

4. The method of claim 1, wherein the IL-2 is recombinant human IL-2 (rhIL-2).

5. The method of claim 1, wherein the anti-TCR Vγ9 monoclonal antibody or antigen binding fragment thereof is chimeric.

6. The method of claim 1, wherein the anti-TCR Vγ9 monoclonal antibody or antigen binding fragment thereof is human or humanized.

7. The method of claim 1, wherein the γδ T cell is a Vγ9⁺ γδ T cell of a Vγ9⁻ γδ T cell.

8. The method of claim 1, wherein the γδ T cells are isolated by flow cytometry, magnetic separation, and negative selection.

9. An isolated γδ T cell produced by the method of claim 8.

10. A method of generating a chimeric antigen receptor (CAR)-γδ T cell, the method comprising:
a. obtaining an isolated γδ T cell of claim 9;
b. contacting the γδ T cell with a nucleic acid encoding a chimeric antigen receptor (CAR), the CAR comprising:
i. an extracellular domain;
ii. a transmembrane domain; and
iii. an intracellular signaling domain,
wherein the CAR optionally further comprises a signal peptide at the amino terminus and a hinge region connecting the extracellular domain and the transmembrane domain, and wherein contacting the γδ T cell with the nucleic acid encoding the CAR generates a CAR γδ T cell.

11. The method of claim 10, wherein the CAR comprises:
i. an extracellular domain comprising an antigen binding domain and/or an antigen binding fragment;
ii. a transmembrane domain comprising a CD8α transmembrane domain;
iii. an intracellular signaling domain comprising a CD3ζ or 4-1BB intracellular domain;
iv. a signal peptide comprising a CD8α signal peptide; and
v. a hinge region comprising a CD8α hinge region.

12. The method of claim 11, wherein the CAR comprises:
i. the transmembrane domain having an amino acid sequence at least 90% identical to SEQ ID NO:1;
ii. the intracellular domain having an amino acid sequence at least 90% identical to SEQ ID NO:2 or SEQ ID NO:3;
iii. the signal peptide having an amino acid sequence at least 90% identical to SEQ ID NO:4; and
iv. the hinge region having an amino acid sequence at least 90% identical to SEQ ID NO:5.

13. The method of claim 10, wherein the extracellular domain comprises an antigen binding domain and/or an antigen binding fragment that specifically binds a tumor antigen.

14. A CAR-γδ T cell produced by the method of claim 10.

15. A pharmaceutical composition comprising the CAR-γδ T cell of claim 14 and a pharmaceutically acceptable carrier.

16. A method of treating or preventing a disease or condition in a subject in need thereof, the method comprising administering a therapeutically effective amount of the pharmaceutical composition of claim 15.

17. The method of claim 16, wherein the disease or condition is cancer.

18. The method of claim 17, wherein the cancer is selected from a solid cancer or a liquid cancer.

19. The method of claim 18, wherein the cancer is selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), a Hodgkin's lymphoma/disease (HD), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

20. The method of claim 16, wherein the disease or condition is an autoimmune disease.

21. The method of claim 20, wherein the autoimmune disease is selected from the group consisting of alopecia, amyloidosis, ankylosing spondylitis, Castleman disease (CD), celiac disease, crohn's disease, endometriosis, fibromyalgia, glomerulonephritis, Graves' disease, Guillain-Barre syndrome, IgA nephropathy, lupus, lyme disease, Meniere's disease, multiple sclerosis, narcolepsy, neutropenia, psoriasis, psoriatic arthritis, rheumatoid arthritis, sarcoidosis, scleroderma, type 1 diabetes, ulcerative colitis, and vitiligo.

22. A method of producing a pharmaceutical composition comprising a CAR-γδ T cell, wherein the methods comprises combining the CAR-γδ T cell of claim 14 with a pharmaceutically acceptable carrier to obtain the pharmaceutical composition.
